(19) United States
(12) Patent Application Publication
(10) Pub. No.: US 2017/0266100 A1
(43) Pub. Date: Sep. 21, 2017

(54) AGENTS AND METHODS FOR THE TEMPORARY SHAPING OF KERATIN-CONTAINING FIBERS

(71) Applicant: Henkel AG & Co. KGaA, Duesseldorf (DE)

(72) Inventors: Thorsten Knappe, Schenefeld (DE); Rolf Bayersdoerfer, Hamburg (DE); Thomas Foerster, Duesseldorf (DE)

(73) Assignee: Henkel AG & Co. KGaA, Duesseldorf (DE)

(21) Appl. No.: 15/615,085

(22) Filed: Jun. 6, 2017

Related U.S. Application Data
(63) Continuation of application No. PCT/EP2015/075376, filed on Nov. 2, 2015.

(30) Foreign Application Priority Data
Dec. 10, 2014 (DE) 10 2014 225 420.5

(51) Int. Cl. A61K 8/86; A61K 8/20; A61K 8/46

(52) U.S. Cl. CPC A61K 8/86; A61K 8/463; A61K 8/20; C01F 5/30

(57) ABSTRACT

The invention relates to a cosmetic product, comprising a) a cosmetic preparation, including, in relation to its total weight, a1) 65 to 96 wt % of at least one polar solvent; a2) 0.1 to 34 wt % of at least one inorganic salt; b) a device for flash evaporation of the cosmetic preparation a), and to a method using corresponding products and to the use of the cosmetic preparation a) as process material in a device for flash evaporation.

### FIELD OF THE INVENTION

The present invention generally relates to the technical field of the temporary reshaping of keratin-containing fibers, in particular human hair. The subject of the invention concerns specific hair cosmetic formulations, which are suitable for application to keratin-containing fibers by means of a flash evaporation method. Moreover, the use of said hair cosmetic formulations in devices for flash evaporation and methods for the temporary reshaping of keratin-containing fibers are the subject of the present invention.

### BACKGROUND OF THE INVENTION

Today, an attractive hairstyle is generally regarded as an essential part of a well-groomed appearance. In this regard, based on current fashion trends, hairstyles time and again are considered chic that for many hair types can only be formed or sustained over a longer period up to several days by the use of active setting substances. Hair treatment agents used for a permanent or temporary shaping of hair therefore play an important role. Whereas during permanent reshaping the chemical structure of the keratin-containing fibers is modified by reduction and oxidation, such modifications of the chemical structure do not occur in temporary reshaping. Suitable agents for temporary shaping typically include synthetic polymers and/or waxes as an active setting substance.

One of the important properties of an agent for the temporary shaping of keratinic fibers, also called a styling agent hereinafter, is to give the treated fibers the strongest possible hold in the new modelled shape, i.e., a shape imposed on the fibers. If the keratinic fibers are human hair, then one also speaks of a strong hairstyle hold or of a high degree of hold of the styling agent. The hairstyle hold is determined substantially by the type and amount of the employed active setting substances, but the other components of the styling agent and the application form may also have an effect.

In the field of the temporary shaping of keratinic fibers, the spray application of suitable cosmetic preparations is of great importance, wherein the preparations are usually applied as pump sprays or aerosol sprays. For this purpose, the cosmetic preparations are packaged in a dispensing device, from which they are sprayed either by mechanical force or with the aid of a propellant via a valve. Both methods have obvious disadvantages. Whereas pump sprays are generally not suitable for a long-lasting uniform spray application of hair cosmetic preparations, aerosol sprays are based on the use of propellants or propellant gases, which, on the one hand, exert no cosmetic effect and which, on the other, can pose a danger to the user when handled improperly.

Against this background, there is a need for alternative approaches to the spraying of hair cosmetic preparations. Flash evaporation has proven to be an alternative spray method of this kind. In this method, which is described, for example, in the international patent application WO 200183071 A1 (Henkel), a liquid or paste-form, solvent-containing composition is heated in an enclosed space to a temperature above the boiling point of the solvent, as a result of which excess pressure is created in the composition. Upon release (throttling) of the pressure, the liquid evaporates and can then be atomized, for example, by a suitable nozzle.

Even if flash evaporation is, therefore, basically suitable for the spray application of hair cosmetic preparations, thus at the same time not every hair cosmetic preparation agent can be atomized by a flash evaporation method. This is due, on the one hand, to the heating of the cosmetic preparation necessary for flash evaporation and, on the other, to the specifics of the spray mist produced by flash evaporation, for example, the produced droplet size and droplet density in the spray mist.

### BRIEF SUMMARY OF THE INVENTION

An object of the present invention, therefore, was to provide specific hair cosmetic preparations for the temporary shaping of keratin-containing fibers, which based on their chemical and physical properties are suitable for the targeted spray application using a device for flash evaporation. Furthermore, the preparations are to be suitable for realizing a high degree of hold, in particular a high degree of long-lasting hold, and a high-volume effect after application by a flash method. It emerged that of the many known preparations effective as hair cosmetics, aqueous or aqueous/alcoholic preparations in particular including an inorganic salt as the setting component are suitable for achieving said object.

A first subject of the present invention, therefore, is a cosmetic product comprising

a) a cosmetic preparation including, based on its total weight,
a1) 65 to 96% by weight of at least one polar solvent,
a2) 0.1 to 34% by weight of at least one inorganic salt;

b) a device for the flash evaporation of cosmetic preparation a).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Cosmetic preparation a) is preferably liquid. Cosmetic preparation a) can be present as a solution or dispersion, for example, as an emulsion or suspension. Preferred cosmetic preparations a) are present in the form of a solution or suspension.

The cosmetic preparation of the invention includes 65 to 96% by weight of at least one polar solvent a1) as the first essential component. Preferred cosmetic products are characterized in that the weight proportion of polar solvent a1) in terms of the total weight of cosmetic preparation a) is 75 to 94% by weight, preferably 85 to 92% by weight. Corresponding agents are characterized by a good cosmetic effect with simultaneously good appliability.

To improve the application properties of the cosmetic preparations of the invention while simultaneously minimizing the thermal stress on possible active or auxiliary substances during the flash evaporation process, it has emerged as advantageous to use polar solvents a1) that have a boiling point (20° C., 1013 mbar) between 50 and 110° C., preferably between 70 and 105° C. In this case, ethanol, isopropanol, and water have proven to be particularly suitable and for this reason these are preferred as polar solvents a1).

Particularly preferred polar solvents a1) or solvent systems are
polar solvents a1), comprising more than 80% by weight, preferably more than 88% by weight, and in particular more than 92% by weight of water, based in each case on the total weight of the polar solvent;
polar solvent a1), comprising, based in each case on the total weight of the polar solvent, at least 60% by weight, preferably at least 80% by weight, particularly preferably at least 90% by weight, and in particular at least 95% by weight of water and ethanol;
polar solvent a1), comprising water and ethanol, the weight ratio of water to ethanol being 5:1 to 1:5, preferably 2:1 to 1:2, and in particular 5:4 to 4:5.

Very particularly preferred cosmetic preparations are characterized in that the proportion of volatile components is a maximum of 55% by weight. The group of these volatile components also include, among others, the polar solvent ethanol. Very particularly preferred cosmetic preparations accordingly in other words are characterized in that the weight proportion of ethanol in terms of the total weight of the cosmetic preparation is a maximum of 55% by weight, preferably 10 to 55% by weight, particularly preferably 25 to 55% by weight, and in particular 40 to 55% by weight.

A second essential component of the cosmetic compositions of the invention is inorganic salt a2). It has proven to be advantageous in regard to the producibility, appliability, and cosmetic effect of the cosmetic compositions of the invention, if the weight proportion of inorganic salt a2) in terms of the total weight of the cosmetic preparation a) is 1.0 to 24% by weight, preferably 2.0 to 14% by weight. Particularly preferred are salt contents above 3.0% by weight, preferably 4.0% by weight, and in particular 5.0% by weight.

Preferred inorganic salts a2) are selected from the chlorides and sulfates of alkali and alkaline earth metals. Preferred salts a2) are, for example, sodium chloride, sodium sulfate, magnesium chloride, and magnesium sulfate. Particularly preferred salts a2) are magnesium sulfate, preferably magnesium heptahydrate MgSO₄.7H₂O, and magnesium chloride MgCl₂. A further particularly preferred salt a2) is sodium chloride NaCl.

In a preferred embodiment, inorganic salt a2) comprises a combination of magnesium sulfate heptahydrate and sodium chloride, wherein the weight ratio of magnesium sulfate heptahydrate to sodium chloride is primarily 1:2 to 50:1, preferably 1:1 to 25:1, and in particular 2:1 to 10:1.

The cosmetic products of the invention, furthermore, comprise, apart from cosmetic preparation a), a device for flash evaporation. The term “flash evaporation” in the context of the present invention describes the creation of vapor when pressure is reduced in a closed space filled with a liquid and having an excess pressure (relative to the environment). A suitable excess pressure can be produced, for example, by heating an amount of cosmetic preparation a) to a temperature T₁ in an enclosed space. In the closed space, the liquid at the particular temperature T₁ has a pressure, the saturation pressure p₁. If the closed space is opened, for example, by a valve to a relaxation space that is not at excess pressure but has the pressure p₀<p₁, then the pressure in the previously closed space is reduced and cosmetic preparation a) or the solvent included in the cosmetic preparation or parts of said solvent evaporate within the scope of the spread of the new pressure level. The forming vapor or spray mist can be used for applying specific cosmetic preparations.

If cosmetic preparation a) is therefore heated in a closed space proceeding from standard conditions (T₀=25° C., p₀=1.000 bar), thus an increased pressure of cosmetic preparation a) also results, in addition to an increased temperature. This increased pressure can be relieved in a relaxation space to a pressure p₀, for example, the ambient air pressure (p₀=1.000 bar), as a result of which evaporation of cosmetic preparation a) is achieved at least partially.

The pressure of cosmetic preparation a) can be relieved directly in the space in which it was previously heated. However, the heated cosmetic preparation a) at excess pressure can be transported alternatively after heating as well to a second chamber in which the pressure release then occurs.

Flash evaporation, in other words, is a method in which cosmetic preparation a) is heated by a heating device in a closed container to temperatures above the ambient temperature, wherein a pressure above the ambient pressure forms in the container and the heated cosmetic preparation a) under increased pressure is then released from the container into the environment.

A device for flash evaporation accordingly is a device that comprises a container and a heating device and is designed such that a cosmetic preparation a) can be heated in the closed container by the heating device to temperatures above the ambient temperature in a way that a pressure above the ambient pressure arises in the container and the heated cosmetic preparation a) under increased pressure can be released from the container into the environment.

Concurrently with or after the pressure release, cosmetic preparation a) can be supplied to a nozzle which can influence, for example, the properties of the vapor or spray mist produced by the flash evaporation, in particular the droplet size or the droplet density but also the spray width and the shape of the spray cone. The use of nozzles, preferably atomizing nozzles, is therefore preferable. The specific nozzle type or the specific nozzle design is selectively established depending on the particular spray mist properties.

In summary, a preferred device for flash evaporation has
b1) a container b1) which is to be opened and closed by means of a valve and defines the closed interior space in which the cosmetic preparation can be taken up,
b2) a heating device b2) which makes it possible to heat the cosmetic preparation located in container b1).

Particularly preferred is the use of an additional nozzle b3), which enables an atomization of cosmetic preparation a) escaping from the container. As an alternative to a valve, a similarly acting closing element, which can close or release an associated opening in the container by an appropriate change in position, can also be used.

A preferred subject of the present invention is a cosmetic product comprising

a) a cosmetic preparation including, based on its total weight,
a1) 65 to 96% by weight of at least one polar solvent,
a2) 0.1 to 34% by weight of at least one inorganic salt;

b) a device for the flash evaporation of cosmetic preparation a), wherein the device for flash evaporation comprises a container b1) and a heating device b2 and is designed such that
cosmetic preparation a) can be taken up in the interior space of container b1),
the interior space of container b1), said space filled at least partially with cosmetic preparation a), can be closed,
cosmetic preparation a) can be heated in the closed interior space of container b1) by means of heating device b2) with an increase in pressure.

A particularly preferred subject of the present invention, therefore, is a cosmetic product comprising
a) a cosmetic preparation including, based on its total weight,
a1) 65 to 96% by weight of at least one polar solvent,

a2) 0.1 to 34% by weight of at least one inorganic salt;

b) a device for the flash evaporation of cosmetic preparation a), comprising
b1) a container b1) to be closed and opened by means of a valve,
b2) a heating device which makes it possible to heat the cosmetic preparation located in container b1),
b3) a nozzle b3) which enables an atomization of cosmetic preparation a).

A particularly preferred subject of the present invention, in other words, is a cosmetic product comprising

a) a cosmetic preparation including, based on its total weight,
a1) 65 to 96% by weight of at least one polar solvent,
a2) 0.1 to 34% by weight of at least one inorganic salt;

b) a device for the flash evaporation of cosmetic preparation a), wherein the device for flash evaporation comprises a container b1) and a heating device b2 and is designed such that
cosmetic preparation a) can be taken up in the interior space of container b1),
the interior space of container b1), said space filled at least partially with cosmetic preparation a), can be closed,
cosmetic preparation a) can be heated in the closed interior space of container b1) by means of heating device b2) with an increase in pressure,
the heated cosmetic preparation a) can be released from the interior space of container b1) into the environment with a reduction in pressure.

Container b1), in which the cosmetic preparation is heated, is designed in a way that makes it possible to close off completely said container from the environment during the heating of cosmetic preparation a) and to open it after the heating, so as to enable flash evaporation of cosmetic preparation a). This can be assured, for example, by a structural element for flow control, in particular a valve.

Container b1), in which the cosmetic preparation is heated, is preferably in contact with another container from which the cosmetic preparation amount provided for flash evaporation is transferred before heating into container b1). The access between this reservoir and container b1) in this case is to be opened and closed by a suitable device, for example, a valve. This additional container is preferably made in the form of a reservoir; i.e., it comprises preferably a multiple, for example, more than 10 times, preferably more than 50 times, of the amount of the cosmetic preparation necessary for an evaporation process. In other words, the additional container/reservoir preferably has a multiple, for example, more than 10 times the volume, preferably more than 20 times, and in particular more than 50 times the volume of container b1).

A further particularly preferred subject of the present invention, therefore, is a cosmetic product comprising

a) a cosmetic preparation including, based on its total weight,
a1) 65 to 96% by weight of at least one polar solvent,
a2) 0.1 to 34% by weight of at least one inorganic salt;

b) a device for the flash evaporation of cosmetic preparation a), comprising
b1) a container b1) to be closed and opened by means of a valve,
b2) a heating device which makes it possible to heat the cosmetic preparation located in closed container b1),
b3) a nozzle b3) which enables an atomization of cosmetic preparation a);

c) a reservoir for cosmetic preparation a), from which cosmetic preparation a) can enter into container b1), wherein
the access between the reservoir and container b1) has a structural element for flow regulation by which the flow of cosmetic preparation a) from the reservoir into container b1) can be interrupted;
the reservoir has at least 10 times the volume, preferably at least 20 times, and in particular at least 50 times the volume of container b1).

The reservoir is not a pressurized container and the cosmetic composition in the reservoir is not under pressure; in other words, the pressure in the interior of the reservoir corresponds to the ambient pressure (also air pressure or atmospheric pressure). Thus, corresponding cosmetic products do not comprise, for example, any propellant. The cosmetic product also does not have a pump device, which is suitable for releasing or spraying the cosmetic preparation into the environment without the action of the device for flash evaporation.

A very particularly preferred subject of the present invention, therefore, is a cosmetic product comprising

a) a cosmetic preparation including, based on its total weight,
a1) 65 to 96% by weight of at least one polar solvent,
a2) 0.1 to 34% by weight of at least one inorganic salt;

b) a device for the flash evaporation of cosmetic preparation a), comprising
b1) a container b1) to be closed and opened by means of a valve,
b2) a heating device which makes it possible to heat the cosmetic preparation located in closed container b1),
b3) a nozzle b3) which enables an atomization of cosmetic preparation a);

c) a reservoir for cosmetic preparation a), from which cosmetic preparation a) can enter into container b1), wherein
the access between the reservoir and container b1) has a structural element for flow regulation by which the flow of cosmetic preparation a) from the reservoir into container b1) can be interrupted;
the reservoir has at least 10 times the volume, preferably at least 50 times the volume of container b1;
the pressure in the interior of the reservoir corresponds to the ambient pressure.

A very particularly preferred subject of the present invention, therefore, is a cosmetic product comprising

a) a cosmetic preparation including, based on its total weight,
a1) 65 to 96% by weight of at least one polar solvent,
a2) 0.1 to 34% by weight of at least one inorganic salt;

b) a device for the flash evaporation of cosmetic preparation a), comprising
b1) a container b1) to be closed and opened by means of a valve,
b2) a heating device which makes it possible to heat the cosmetic preparation located in closed container b1),
b3) a nozzle b3) which enables an atomization of cosmetic preparation a,

c) a reservoir for cosmetic preparation a), from which cosmetic preparation a) can enter into container b1), wherein
the access between the reservoir and container b1) has a structural element for flow regulation by which the flow of cosmetic preparation a) from the reservoir into container b1) can be interrupted;
the reservoir has at least 10 times the volume, preferably at least 50 times the volume of container b1;
the pressure in the interior of the reservoir corresponds to the ambient pressure and the cosmetic product comprises no propellant.

Preferred furthermore are cosmetic products, comprising

a) a cosmetic preparation including, based on its total weight,
a1) 65 to 96% by weight of at least one polar solvent,
a2) 0.1 to 34% by weight of at least one inorganic salt;

b) a device for the flash evaporation of cosmetic preparation a), comprising
b1) a container b1) to be closed and opened by means of a valve,
b2) a heating device which makes it possible to heat the cosmetic preparation located in closed container b1),
b3) a nozzle b3) which enables an atomization of cosmetic preparation a);

c) a reservoir for cosmetic preparation a), from which cosmetic preparation a) can enter into container b1), wherein
the access between the reservoir and container b1) has a structural element for flow regulation by which the flow of cosmetic preparation a) from the reservoir into container b1) can be interrupted;
the reservoir has at least 10 times the volume, preferably at least 50 times the volume of container b1;
the pressure in the interior of the reservoir corresponds to the ambient pressure,
wherein the cosmetic product does not have a pump device that is suitable for releasing or spraying cosmetic preparation a) without the action of the device for flash evaporation.

In summary, a particularly preferred subject of the present invention, therefore, is a cosmetic product comprising

a) a cosmetic preparation including, based on its total weight,
a1) 65 to 96% by weight of at least one polar solvent,
a2) 0.1 to 34% by weight of at least one inorganic salt;

b) a device for the flash evaporation of cosmetic preparation a), comprising
b1) a container b1) to be closed and opened by means of a valve,
b2) a heating device which makes it possible to heat the cosmetic preparation located in closed container b1),
b3) a nozzle b3) which enables an atomization of cosmetic preparation a);

c) a reservoir for cosmetic preparation a), from which cosmetic preparation a) can enter into container b1), wherein
the access between the reservoir and container b1) has a structural element for flow regulation by which the flow of cosmetic preparation a) from the reservoir into container b1) can be interrupted;
the reservoir has at least 10 times the volume, preferably at least 50 times the volume of container b1;
the pressure in the interior of the reservoir corresponds to the ambient pressure and the cosmetic product comprises no propellant,
wherein the cosmetic product does not have a pump device that is suitable for releasing or spraying cosmetic preparation a) without the action of the device for flash evaporation.

Apart from the two previously described components a1) and a2), cosmetic preparation a) of the invention can include further active or auxiliary substances, wherein active or auxiliary substances of this kind that improve the producibility, appliability, and/or the cosmetic action of the cosmetic preparations of the invention are preferred in particular.

To improve the producibility, appliablity, and cosmetic effect, cosmetic preparation a) preferably includes nonionic surfactant a3), wherein particularly preferred cosmetic preparations a) are characterized in that, based on their total weight, they include 0.05 to 4.0% by weight, preferably 0.1 to 2.0% by weight, and in particular 0.2 to 1.0% by weight of nonionic surfactant a3).

Preferred nonionic surfactants are PEG derivatives of hydrogenated castor oil, which can be obtained, e.g., under the name PEG Hydrogenated Castor Oil, e.g., PEG-30 Hydrogenated Castor Oil, PEG-33 Hydrogenated Castor Oil, PEG-35 Hydrogenated Castor Oil, PEG-36 Hydrogenated Castor Oil, PEG-40 Hydrogenated Castor Oil, or PEG-60 Hydrogenated Castor Oil. Particularly preferred according to the invention are nonionic surfactants selected from the group of PEG derivatives of hydrogenated castor oil, particularly preferably from the group of PEG-40 Hydrogenated Castor Oil and PEG-60 Hydrogenated Castor Oil.

Polyalkylene glycols a4), in particular polyethylene glycols, form a second group of preferred ingredients of cosmetic preparations a). It has proven advantageous for the cosmetic effect of the preparations of the invention, if, based on their total weight, they include 0.1 to 10% by weight, preferably 0.2 to 8.0% by weight, and in particular 0.5 to 5.0% by weight of a polyalkylene glycol a4). The group of polyalkylene glycols include, for example, polyethylene glycols and the particularly preferred polypropylene glycols.

Suitable according to the invention are liquid and solid polyethylene glycols (PEG), for example, polyalkylene glycols with molar masses of 250 (PEG-4) to 3350 (PEG-75). Particularly preferred are polyethylene glycols with the INCI name: PEG-6, PEG-7, PEG-8, PEG-9, PEG-10, PEG-12, PEG-14, PEG-16, PEG-18, PEG-20, PEG-32, and PEG-40. Polyethylene glycols with the INCI name PEG-8 and PEG-32 are particularly preferred.

A third group of preferred active and auxiliary substances is formed by film-forming polymers a5), whose weight proportion in terms of the total weight of cosmetic preparation a) if used, is 0.1 to 10% by weight, preferably 0.2 to 8.0% by weight, and in particular 0.5 to 5.0% by weight.

Permanently and temporarily cationic, anionic, nonionic, or amphoteric polymers as well are suitable as film-forming polymers a5). These film-forming polymers can be synthetic or natural in origin.

Examples of customary film-forming polymers a5) are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/ltaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate, and Styrene/VP Copolymer.

The vinylpyrrolidone homo- or copolymers form a first group of particularly preferred polymers a5). Polymers used with particular preference are:
polyvinylpyrrolidones (INCI name PVP; CAS number 9003-39-8), as they are marketed, for example, under the name Luviskol® (BASF);
copolymers of vinylpyrrolidone and vinyl acetate (INCI name VP/VA Copolymer; CAS number: 25086-89-9), as they are marketed, for example, under the name Luviskol® VA (BASF);
copolymers of vinylpyrrolidone and dimethylaminopropyl methacrylamide (INCI name VP/DMAPA Acrylates Copolymer; CAS number: 175893-71-7), as they are marketed, for example, under the name Styleze® CC-10 (Ashland);
copolymers of vinylpyrrolidone with vinyl caprolactam and dimethylaminoethyl methacrylate (INCI name: Vinyl Caprolactam/VP/Dimethylaminoethyl Methacrylate Copolymer; CAS number: 102972-64-5), as they are marketed, for example, under the name Advantage® LC (Ashland).

Because of their cosmetic action in combination with inorganic salts a2), film-forming polymers used preferentially according to the invention are selected in particular from the group of vinylpyrrolidone homo- and copolymers, preferably from the group of polyvinylpyrrolidones and the copolymers of vinylpyrrolidone and vinyl acetate, preferably vinylpyrrolidone/vinyl acetate copolymers.

In particular, additional care substances can be named as further suitable active or auxiliary substances.

The agent can include as a care substance, for example, at least one protein hydrolysate and/or a derivative thereof. Protein hydrolysates are product mixtures obtained by acid-, base-, or enzyme-catalyzed degradation of proteins. The term ‘protein hydrolysates’ according to the invention is also understood to mean total hydrolysates, as well as individual amino acids and derivatives thereof, and mixtures of different amino acids. The molar weight of protein hydrolysates usable according to the invention is between 75 (the molar weight of glycine) and 200,000; the molar weight is preferably 75 to 50,000 daltons, and very particularly preferably 75 to 20,000 daltons.

The agent of the invention can include further at least one vitamin, provitamin, vitamin precursor, and/or a derivative thereof as a care substance. In this case, the vitamins, provitamins, and vitamin precursors of this kind that are usually assigned to the groups A, B, C, E, F, and H are preferred according to the invention.

Other care substances are panthenol, caffeine, nicotinamide, and sorbitol.

The agents of the invention, furthermore, can include at least one plant extract but also mono- or oligosaccharides and/or lipids as a care substance.

The composition of some particularly preferred cosmetic preparations of the invention can be obtained from the following tables (the data are given in percentages by weight, based on the total weight of the cosmetic agent, unless otherwise stated). The statements made previously regarding cosmetic preparations a) of the invention apply mutatis mutandis with respect to other preferred embodiments of these particularly preferred compositions.

| | Formula 1 | Formula 2 | Formula 3 | Formula 4 | Formula 5 |
|---|---|---|---|---|---|
| Polar solvent a1) | 65 to 96 | 75 to 94 | 85 to 92 | 88 | 91 |
| Inorganic salt a2) | 0.1 to 34 | 1.0 to 24 | 2.0 to 14 | 11 | 3.0 |
| Optional additives | To 100 | To 100 | To 100 | To 100 | To 100 |
| | Formula 6 | Formula 7 | Formula 8 | Formula 9 | Formula 10 |
| Water | 65 to 96 | 75 to 94 | 85 to 92 | 88 | 91 |
| Inorganic salt a2) | 0.1 to 34 | 1.0 to 24 | 2.0 to 14 | 11 | 3.0 |
| Optional additives | To 100 | To 100 | To 100 | To 100 | To 100 |
| | Formula 11 | Formula 12 | Formula 13 | Formula 14 | Formula 15 |
| Water, ethanol | 65 to 96 | 75 to 94 | 85 to 92 | 88 | 91 |
| Inorganic salt a2) | 0.1 to 34 | 1.0 to 24 | 2.0 to 14 | 11 | 3.0 |
| Optional additives | To 100 | To 100 | To 100 | To 100 | To 100 |
| | Formula 16 | Formula 17 | Formula 18 | Formula 19 | Formula 20 |
| Polar solvent a1) | 65 to 96 | 75 to 94 | 85 to 92 | 88 | 91 |
| Sodium chloride | 0.1 to 34 | 1.0 to 24 | 2.0 to 14 | 11 | 3.0 |
| Optional additives | To 100 | To 100 | To 100 | To 100 | To 100 |
| | Formula 21 | Formula 22 | Formula 23 | Formula 24 | Formula 25 |
| Water | 65 to 96 | 75 to 94 | 85 to 92 | 88 | 91 |
| Sodium chloride | 0.1 to 34 | 1.0 to 24 | 2.0 to 14 | 11 | 3.0 |
| Optional additives | To 100 | To 100 | To 100 | To 100 | To 100 |
| | Formula 26 | Formula 27 | Formula 28 | Formula 29 | Formula 30 |
| Water, ethanol | 65 to 96 | 75 to 94 | 85 to 92 | 88 | 91 |
| Sodium chloride | 0.1 to 34 | 1.0 to 24 | 2.0 to 14 | 11 | 3.0 |
| Optional additives | To 100 | To 100 | To 100 | To 100 | To 100 |
| | Formula 31 | Formula 32 | Formula 33 | Formula 34 | Formula 35 |
| Polar solvent a1) | 65 to 96 | 75 to 94 | 85 to 92 | 88 | 91 |
| Magnesium sulfate | 0.1 to 34 | 1.0 to 24 | 2.0 to 14 | 11 | 3.0 |
| Optional additives | To 100 | To 100 | To 100 | To 100 | To 100 |
| | Formula 36 | Formula 37 | Formula 38 | Formula 39 | Formula 40 |
| Water | 65 to 96 | 75 to 94 | 85 to 92 | 88 | 91 |
| Magnesium sulfate | 0.1 to 34 | 1.0 to 24 | 2.0 to 14 | 11 | 3.0 |
| Optional additives | To 100 | To 100 | To 100 | To 100 | To 100 |
| | Formula 41 | Formula 42 | Formula 43 | Formula 44 | Formula 45 |
| Water, ethanol | 65 to 96 | 75 to 94 | 85 to 92 | 88 | 91 |
| Magnesium sulfate | 0.1 to 34 | 1.0 to 24 | 2.0 to 14 | 11 | 3.0 |
| Optional additives | To 100 | To 100 | To 100 | To 100 | To 100 |
| | Formula 46 | Formula 47 | Formula 48 | Formula 49 | Formula 50 |
| Polar solvent a1) | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Optional additives | To 100 | To 100 | To 100 | To 100 | To 100 |
| | Formula 51 | Formula 52 | Formula 53 | Formula 54 | Formula 55 |
| Water | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Optional additives | To 100 | To 100 | To 100 | To 100 | To 100 |
| | Formula 56 | Formula 57 | Formula 58 | Formula 59 | Formula 60 |
| Water, ethanol | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Optional additives | To 100 | To 100 | To 100 | To 100 | To 100 |
| | Formula 61 | Formula 62 | Formula 63 | Formula 64 | Formula 65 |
| Polar solvent a1) | 65 to 96 | 75 to 94 | 85 to 92 | 88 | 91 |
| Inorganic salt a2) | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula 66 | Formula 67 | Formula 68 | Formula 69 | Formula 70 |
| Water | 65 to 96 | 75 to 94 | 85 to 92 | 88 | 91 |
| Inorganic salt a2) | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula 71 | Formula 72 | Formula 73 | Formula 74 | Formula 75 |
| Water, ethanol | 65 to 96 | 75 to 94 | 85 to 92 | 88 | 91 |
| Inorganic salt a2) | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula 76 | Formula 77 | Formula 78 | Formula 79 | Formula 80 |
| Polar solvent a1) | 65 to 96 | 75 to 94 | 85 to 92 | 88 | 91 |
| Sodium chloride | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula 81 | Formula 82 | Formula 83 | Formula 84 | Formula 85 |
| Water | 65 to 96 | 75 to 94 | 85 to 92 | 88 | 91 |
| Sodium chloride | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula 86 | Formula 87 | Formula 88 | Formula 89 | Formula 90 |
| Water, ethanol | 65 to 96 | 75 to 94 | 85 to 92 | 88 | 91 |
| Sodium chloride | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula 91 | Formula 92 | Formula 93 | Formula 94 | Formula 95 |
| Polar solvent a1) | 65 to 96 | 75 to 94 | 85 to 92 | 88 | 91 |
| Magnesium sulfate | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | | | | | Formula |
| | Formula 96 | Formula 97 | Formula 98 | Formula 99 | 100 |
| Water | 65 to 96 | 75 to 94 | 85 to 92 | 88 | 91 |
| Magnesium sulfate | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula | Formula | Formula | Formula | Formula |
| | 101 | 102 | 103 | 104 | 105 |
| Water, ethanol | 65 to 96 | 75 to 94 | 85 to 92 | 88 | 91 |
| Magnesium sulfate | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula | Formula | Formula | Formula | Formula |
| | 106 | 107 | 108 | 109 | 110 |
| Polar solvent a1) | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula | Formula | Formula | Formula | Formula |
| | 111 | 112 | 113 | 114 | 115 |
| Water | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula | Formula | Formula | Formula | Formula |
| | 116 | 117 | 118 | 119 | 120 |
| Water, ethanol | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Nonionic surfactant a3) | 0.05 to 4.0 | 0.1 to 2.0 | 0.2 to 1.0 | 0.2 | 0.4 |
| Optional additives | To 100 | To 100 | To 100 | To 100 | To 100 |
| | Formula | Formula | Formula | Formula | Formula |
| | 121 | 122 | 123 | 124 | 125 |
| Polar solvent a1) | 65 to 96 | 75 to 94 | 85 to 92 | 88 | 91 |
| Inorganic salt a2) | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 126 | 127 | 128 | 129 | 130 |
| Water | 65 to 96 | 75 to 94 | 85 to 92 | 88 | 91 |
| Inorganic salt a2) | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 131 | 132 | 133 | 134 | 135 |
| Water, ethanol | 65 to 96 | 75 to 94 | 85 to 92 | 88 | 91 |
| Inorganic salt a2) | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 136 | 137 | 138 | 139 | 140 |
| Polar solvent a1) | 65 to 96 | 75 to 94 | 85 to 92 | 88 | 91 |
| Sodium chloride | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 141 | 142 | 143 | 144 | 145 |
| Water | 65 to 96 | 75 to 94 | 85 to 92 | 88 | 91 |
| Sodium chloride | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 146 | 147 | 148 | 149 | 150 |
| Water, ethanol | 65 to 96 | 75 to 94 | 85 to 92 | 88 | 91 |
| Sodium chloride | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 151 | 152 | 153 | 154 | 155 |
| Polar solvent a1) | 65 to 96 | 75 to 94 | 85 to 92 | 88 | 91 |
| Magnesium sulfate | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 156 | 157 | 158 | 159 | 160 |
| Water | 65 to 96 | 75 to 94 | 85 to 92 | 88 | 91 |
| Magnesium sulfate | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 161 | 162 | 163 | 164 | 165 |
| Water, ethanol | 65 to 96 | 75 to 94 | 85 to 92 | 88 | 91 |
| Magnesium sulfate | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 166 | 167 | 168 | 169 | 170 |
| Polar solvent a1) | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 171 | 172 | 173 | 174 | 175 |
| Water | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 176 | 177 | 178 | 179 | 180 |
| Water, ethanol | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 181 | 182 | 183 | 184 | 185 |
| Polar solvent a1) | 65 to 96 | 75 to 94 | 85 to 92 | 88 | 91 |
| Inorganic salt a2) | 0.1 to 34 | 1.0 to 24 | 2.0 to 14 | 11 | 3.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 186 | 187 | 188 | 189 | 190 |
| Water | 65 to 96 | 75 to 94 | 85 to 92 | 88 | 91 |
| Inorganic salt a2) | 0.1 to 34 | 1.0 to 24 | 2.0 to 14 | 11 | 3.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 191 | 192 | 193 | 194 | 195 |
| Water, ethanol | 65 to 96 | 75 to 94 | 85 to 92 | 88 | 91 |
| Inorganic salt a2) | 0.1 to 34 | 1.0 to 24 | 2.0 to 14 | 11 | 3.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 196 | 197 | 198 | 199 | 200 |
| Polar solvent a1) | 65 to 96 | 75 to 94 | 85 to 92 | 88 | 91 |
| Sodium chloride | 0.1 to 34 | 1.0 to 24 | 2.0 to 14 | 11 | 3.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 201 | 202 | 203 | 204 | 205 |
| Water | 65 to 96 | 75 to 94 | 85 to 92 | 88 | 91 |
| Sodium chloride | 0.1 to 34 | 1.0 to 24 | 2.0 to 14 | 11 | 3.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 206 | 207 | 208 | 209 | 210 |
| Water, ethanol | 65 to 96 | 75 to 94 | 85 to 92 | 88 | 91 |
| Sodium chloride | 0.1 to 34 | 1.0 to 24 | 2.0 to 14 | 11 | 3.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 211 | 212 | 213 | 214 | 215 |
| Polar solvent a1) | 65 to 96 | 75 to 94 | 85 to 92 | 88 | 91 |
| Magnesium sulfate | 0.1 to 34 | 1.0 to 24 | 2.0 to 14 | 11 | 3.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 216 | 217 | 218 | 219 | 220 |
| Water | 65 to 96 | 75 to 94 | 85 to 92 | 88 | 91 |
| Magnesium sulfate | 0.1 to 34 | 1.0 to 24 | 2.0 to 14 | 11 | 3.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 221 | 222 | 223 | 224 | 225 |
| Water, ethanol | 65 to 96 | 75 to 94 | 85 to 92 | 88 | 91 |
| Magnesium sulfate | 0.1 to 34 | 1.0 to 24 | 2.0 to 14 | 11 | 3.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 226 | 227 | 228 | 229 | 230 |
| Polar solvent a1) | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 231 | 232 | 233 | 234 | 235 |
| Water | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 236 | 237 | 238 | 239 | 240 |
| Water, ethanol | 65 to 96 | 75 to 94 | 85 to 92 | 88 | 91 |
| Sodium chloride, | 0.1 to 34 | 1.0 to 24 | 2.0 to 14 | 11 | 3.0 |
| magnesium sulfate | | | | | |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 241 | 242 | 243 | 244 | 245 |
| Polar solvent a1) | 65 to 96 | 75 to 94 | 85 to 92 | 88 | 91 |
| Inorganic salt a2) | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Polyalkylene glycol a4) | 0.1 to 10 | 0.2 to 8.0 | 0.5 to 5.0 | 0.2 | 2.0 |
| Optional additives | To 100 | To 100 | To 100 | To 100 | To 100 |
| | Formula | Formula | Formula | Formula | Formula |
| | 246 | 247 | 248 | 249 | 250 |
| Polar solvent a1) | 65 to 96 | 75 to 94 | 85 to 92 | 88 | 91 |
| Inorganic salt a2) | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 251 | 252 | 253 | 254 | 255 |
| Polar solvent a1) | 65 to 96 | 75 to 94 | 85 to 92 | 88 | 91 |
| Inorganic salt a2) | 0.1 to 34 | 1.0 to 24 | 2.0 to 14 | 11 | 3.0 |
| Polyalkylene glycol a4) | 0.1 to 10 | 0.2 to 8.0 | 0.5 to 5.0 | 0.2 | 2.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 256 | 257 | 258 | 259 | 260 |
| Polar solvent a1) | 65 to 96 | 75 to 94 | 85 to 92 | 88 | 91 |
| Inorganic salt a2) | 0.1 to 34 | 1.0 to 24 | 2.0 to 14 | 11 | 3.0 |
| Nonionic surfactant a3) | 0.05 to 4.0 | 0.1 to 2.0 | 0.2 to 1.0 | 0.2 | 0.4 |
| Polyalkylene glycol a4) | 0.1 to 10 | 0.2 to 8.0 | 0.5 to 5.0 | 0.2 | 2.0 |
| Film-forming polymer | 0.1 to 10 | 0.2 to 8.0 | 0.5 to 5.0 | 0.2 | 2.0 |
| a5) | | | | | |
| Optional additives | To 100 | To 100 | To 100 | To 100 | To 100 |
| | Formula | Formula | Formula | Formula | Formula |
| | 261 | 262 | 263 | 264 | 265 |
| Water | 65 to 96 | 75 to 94 | 85 to 92 | 88 | 91 |
| Magnesium sulfate | 0.1 to 34 | 1.0 to 24 | 2.0 to 14 | 11 | 3.0 |
| PEG-40 Hydrogenated | 0.05 to 4.0 | 0.1 to 2.0 | 0.2 to 1.0 | 0.2 | 0.4 |
| Castor Oil | | | | | |
| PEG-32 | 0.1 to 10 | 0.2 to 8.0 | 0.5 to 5.0 | 0.2 | 2.0 |
| PVPA/A Copolymer | 0.1 to 10 | 0.2 to 8.0 | 0.5 to 5.0 | 0.2 | 2.0 |
| Optional additives | To 100 | To 100 | To 100 | To 100 | To 100 |

Very particularly preferred cosmetic preparations contain, apart from the above-described components a1) to a5), only small amounts of further active and auxiliary substances. Cosmetic preparations, characterized in that the weight proportion of components a1), a2) and, if present, a3), a4), and/or a5) in terms of the total weight of the cosmetic preparation is at least 80% by weight, preferably at least 90% by weight, particularly preferably at least 95% by weight, are particularly preferred due to their easy producibility and good cosmetic action. Very particularly preferred cosmetic preparations a), based on their total weight, consist of at least 84% by weight, preferably at least 88% by weight, and in particular at least 92% by weight of components a1) and a2).

As set forth above, cosmetic preparations a) of the invention are suitable in a particular way for application by means of a device for flash evaporation. A further subject of the present invention, therefore, is the use of a cosmetic preparation a) including, based on its total weight,
a1) 65 to 96% by weight of at least one polar solvent,
a2) 0.1 to 34% by weight of at least one inorganic salt, as a process material in a device for flash evaporation.

The subject of the present invention, moreover, is the use of a product of the invention for treating keratin-containing fibers, in particular human hair, with a cosmetic preparation a) or for the temporary shaping of keratin-containing fibers, in particular human hair.

A method for the temporary shaping of keratin-containing fibers, in particular human hair, in which the keratinic fibers are acted upon by a device for flash evaporation with a cosmetic preparation a) including, based on its total weight,
a1) 65 to 96% by weight of at least one polar solvent,
a2) 0.1 to 34% by weight of at least one inorganic salt, is a further subject of the present invention. Cosmetic preparation a) is converted by the device for flash evaporation preferably into a spray mist, which subsequently acts upon the keratin-containing fibers.

So as to achieve a sufficient spray effect, cosmetic preparation a) is preferably heated to temperatures above the boiling point of the polar solvent or solvent mixture included in cosmetic preparation a).

If the polar solvent is water or solvent mixtures with a proportion of water above 50% by weight (based on the total weight of the solvent mixture), the cosmetic preparation is preferably heated to temperatures above 100° C., preferably to temperatures of 100° C. and 240° C., particularly preferably to temperatures of 140° C. to 160° C.

The excess pressure, achieved by heating cosmetic preparation a) in the cases in which the polar solvent is water or solvent mixtures with a proportion of water above 50% by weight (based on the total weight of the solvent mixture), is preferably between 1.1 and 8 bar, preferably between 1.2 and 4 bar.

A preferred subject of the invention is a method for changing the color of keratin-containing fibers, in particular human hair, in which the keratin-containing fibers are acted upon by a device for flash evaporation with a cosmetic preparation a) including, based on its total weight,
a1) 65 to 96% by weight of at least one polar solvent,
a2) 0.1 to 34% by weight of at least one inorganic salt,

wherein
a partial amount of cosmetic preparation a) located in a reservoir is transferred from this reservoir, in whose interior a pressure corresponding to the ambient pressure predominates, into a container b1);
thereafter the access between the reservoir and container b1) is interrupted by a structural element for flow regulation by which the flow of cosmetic preparation a) from the reservoir into container b1) can be interrupted;
thereafter cosmetic preparation a) located in container b1), closed off from the environment, is heated by a heating device, so that the pressure in the interior of container b1) increases to values above the ambient pressure, preferably to values between 1.1 and 8 bar, in particular to values between 1.2 and 4 bar;
thereafter container b1), having a pressure above the ambient pressure, is opened in a way that releases the exit of at least one partial amount, preferably at least 50% by weight, preferably at least 80% by weight, and in particular at least 90% by weight of the cosmetic preparation, located in container b1), from container b1) into the environment with a reduction of the pressure predominating in container b1) at the time the container is opened.

The release of cosmetic preparation a) into the environment occurs preferably with the formation of a spray mist of cosmetic preparation a).

Cosmetic preparation a) released from container b1) is preferably applied to keratinic fibers, in particular human hair.

Methods during which the cosmetic preparation released from container b1) is passed through a nozzle before action upon the keratinic fibers are particularly preferred.

The statements made in regard to cosmetic preparations a) of the invention and the device for flash evaporation b) apply mutatis mutandis with respect to further preferred embodiments of the uses of the invention and the method of the invention.

The agents, uses, and methods of the invention and some of their preferred embodiments are characterized in summary by the following points:

A cosmetic product comprising
a) a cosmetic preparation including, based on its total weight,
a1) 65 to 96% by weight of at least one polar solvent,
a2) 0.1 to 34% by weight of at least one inorganic salt;

b) a device for the flash evaporation of cosmetic preparation a).

The cosmetic product according to point 1, characterized in that the device for flash evaporation comprises a container b1) and a heating device b2) and is designed such that
cosmetic preparation a) can be taken up in the interior space of container b1),
the interior space of container b1), said space filled at least partially with cosmetic preparation a), can be closed,
cosmetic preparation a) can be heated in the closed interior space of container b1) by means of heating device b2) with an increase in pressure,
the heated cosmetic preparation a) can be released from the interior space of container b1) into the environment with a reduction in pressure.

The cosmetic product according to one of the preceding points, characterized in that the weight proportion of polar solvent a1) in terms of the total weight of cosmetic preparation a) is 75 to 94% by weight, preferably 85 to 92% by weight.

The cosmetic product according to one of the preceding points, characterized in that polar solvent a1) has a boiling point (20° C., 1013 mbar) between 50 and 110° C., preferably between 70 and 105° C.

The cosmetic products according to one of the preceding points, characterized in that polar solvent a1) is selected from the group comprising ethanol, isopropanol, and water.

The cosmetic products according to one of the preceding points, characterized in that the weight proportion of water and ethanol in terms of the total weight of polar solvent a1) is at least 60% by weight, preferably at least 80% by weight, particularly preferably at least 90% by weight, and in particular at least 95% by weight.

The cosmetic products according to one of the preceding points, characterized in that the weight proportion of water in terms of the total weight of polar solvent a1) is more than 80% by weight, preferably more than 88% by weight, and in particular more than 92% by weight.

The cosmetic products according to one of the preceding points, characterized in that the weight proportion of ethanol in terms of the total weight of the cosmetic preparation is a maximum of 55% by weight, preferably 10 to 55% by weight, particularly preferably 25 to 55% by weight, and in particular 40 to 55% by weight.

The cosmetic products according to one of the preceding points, characterized in that polar solvent a1) comprises water and ethanol and the weight ratio of water to ethanol is 5:1 to 1:5, preferably 2:1 to 1:2, and in particular 5:4 to 4:5.

The cosmetic product according to one of the preceding points, characterized in that the weight proportion of inorganic salt a2) in terms of the total weight of cosmetic preparation a) is 1.0 to 24% by weight, preferably 2.0 to 14% by weight.

The cosmetic product according to one of the preceding points, characterized in that inorganic salt a2) is selected from the group of chlorides and sulfates of alkali and alkaline earth metals, in particular from the group of magnesium sulfate, magnesium chloride, and sodium chloride.

The cosmetic product according to one of the preceding points, characterized in that cosmetic preparation a), based on its total weight, includes 0.05 to 4.0% by weight, preferably 0.1 to 2.0% by weight, and in particular 0.2 to 1.0% by weight of nonionic surfactant a3).

The cosmetic product according to point 12, characterized in that nonionic surfactant a3) is selected from the group of PEG derivatives of hydrogenated castor oil, particularly preferably from the group of PEG-40 Hydrogenated Castor Oil and PEG-60 Hydrogenated Castor Oil.

The cosmetic product according to one of the preceding points, characterized in that cosmetic preparation a), based on its total weight, includes 0.1 to 10% by weight, preferably 0.2 to 8.0% by weight, and in particular 0.5 to 5.0% by weight of a polyalkylene glycol a4).

The cosmetic product according to point 14, characterized in that polyalkylene glycol a4) is selected from the group of polyethylene glycols, preferably polyethylene glycols with the INCI name: PEG-6, PEG-7, PEG-8, PEG-9, PEG-10, PEG-12, PEG-14, PEG-16, PEG-18, PEG-20, PEG-32, and PEG-40.

The cosmetic product according to one of the preceding points, characterized in that cosmetic preparation a), based on its total weight, includes 0.1 to 10% by weight, preferably 0.2 to 8.0% by weight, and in particular 0.5 to 5.0% by weight of a film-forming polymer a5).

The cosmetic product according to point 16, characterized in that film-forming polymer a5) is selected from the group of nonionic polymers, preferably from the group of polyvinylpyrrolidones and vinylpyrrolidone/vinyl acetate copolymers, preferably vinylpyrrolidone/vinyl acetate copolymers.

The cosmetic product according to one of the preceding points, characterized in that cosmetic preparation a), based on its total weight, consists of at least 80% by weight, preferably at least 90% by weight, and in particular at least 95% by weight of components
a1), a2) and, if present, a3), a4), and/or a5).

The cosmetic product according to one of the preceding points, characterized in that cosmetic preparation a), based on its total weight, consists of at least 84% by weight, preferably at least 88% by weight, and in particular at least 92% by weight of components a1) and a2).

Use of a cosmetic preparation a) including, based on its total weight,
a1) 65 to 96% by weight of at least one polar solvent,
a2) 0.1 to 34% by weight of at least one inorganic salt, as a process material in a device for flash evaporation.

The use of a product according to one of points 1 to 19 for treating keratin-containing fibers, in particular human hair, with a cosmetic preparation a).

Use of a product according to one of points 1 to 19 for the temporary shaping of keratin-containing fibers, in particular human hair.

A method for the temporary shaping of keratin-containing fibers, in particular human hair, in which the keratin-containing fibers are acted upon by a device for flash evaporation with a cosmetic preparation a) including, based on its total weight,
a1) 65 to 96% by weight of at least one polar solvent,
a2) 0.1 to 34% by weight of at least one inorganic salt.

The method according to point 23, characterized in that
a partial amount of cosmetic preparation a) located in a reservoir is transferred from this reservoir, in whose interior a pressure corresponding to the ambient pressure predominates, into a container b1);
thereafter the access between the reservoir and container b1) is interrupted by a structural element for flow regulation by which the flow of cosmetic preparation a) from the reservoir into container b1) can be interrupted;
thereafter cosmetic preparation a) located in container b1), closed off from the environment, is heated by a heating device, so that the pressure in the interior of container b1) increases to values above the ambient pressure, preferably to values between 1.1 and 8 bar, in particular to values between 1.2 and 4 bar;
thereafter container b1), having a pressure above the ambient pressure, is opened in a way that releases the exit of at least one partial amount, preferably at least 50% by weight, preferably at least 80% by weight, and in particular at least 90% by weight of the cosmetic preparation, located in container b1), from container b1) into the environment with a reduction of the pressure predominating in container b1) at the time the container is opened.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic product, comprising:
a) a cosmetic preparation including, based on its total weight,
a1) 65 to 96% by weight of at least one polar solvent,
a2) 0.1 to 34% by weight of at least one inorganic salt;
b) a device for the flash evaporation of cosmetic preparation a).

2. The cosmetic product according to claim 1, wherein the device for flash evaporation comprises a container b1) and a heating device b2) and is configured such that
the cosmetic preparation a) can be taken up in the interior space of container b1),
the interior space of container b1), said space filled at least partially with cosmetic preparation a), can be closed,
cosmetic preparation a) can be heated in the closed interior space of container b1) by means of heating device b2) with an increase in pressure,
the heated cosmetic preparation a) can be released from the interior space of container b1) into the environment with a reduction in pressure.

3. The cosmetic product according to claim 1, wherein the weight proportion of polar solvent a1) in terms of the total weight of cosmetic preparation a) is 75 to 94% by weight.

4. The cosmetic product according to claim 1, wherein the weight proportion of polar solvent a1) in terms of the total weight of cosmetic preparation a) 85 to 92% by weight.

5. The cosmetic product according to claim 1, wherein the weight proportion of inorganic salt a2) in terms of the total weight of cosmetic preparation a) is 1.0 to 24% by weight.

6. The cosmetic product according to claim 1, wherein the weight proportion of inorganic salt a2) in terms of the total weight of cosmetic preparation a) is 2.0 to 14% by weight.

7. The cosmetic product according to claim 1, wherein the inorganic salt a2) is selected from the group consisting of chlorides and sulfates of alkali and alkaline earth metals.

8. The cosmetic product according to claim 1, wherein the inorganic salt a2) is selected form the group consisting of magnesium sulfate, magnesium chloride, and sodium chloride.

9. The cosmetic product according to claim 1, wherein cosmetic preparation a), based on its total weight, includes 0.05 to 4.0% by weight of a nonionic surfactant a3).

10. The cosmetic product according to claim 1, wherein cosmetic preparation a), based on its total weight, includes 0.1 to 2.0% by weight of a nonionic surfactant a3).

11. The cosmetic product according to claim 1, wherein cosmetic preparation a), based on its total weight, includes 0.2 to 1.0% by weight of a nonionic surfactant a3).

12. The cosmetic product according to claim 1, wherein cosmetic preparation a), based on its total weight, includes 0.1 to 10% by weight of a polyalkylene glycol a4).

13. The cosmetic product according to claim 1, wherein cosmetic preparation a), based on its total weight, includes 0.2 to 8% by weight of a polyalkylene glycol a4).

14. The cosmetic product according to claim 1, wherein cosmetic preparation a), based on its total weight, includes 0.5 to 5% by weight of a polyalkylene glycol a4).
